(19) United States
(12) Patent Application Publication
(10) Pub. No.: US 2001/0026791 A1
(43) Pub. Date: Oct. 4, 2001

(54) Gel composition and thickened hair treatment composition

(76) Inventors: Michael Lede, Langen (DE); Susanne Birkel, Glashuetten (DE); Michael Franzke, Rossdorf (DE); Miriam Gruenkemeier, Eltville (DE); Hildegard Henze, Darmstadt (DE)

Correspondence Address: STRIKER, STRIKER & STENBY, 103 East Neck Road, Huntington, NY, 11743, US

(21) Appl. No.: 09/788,949

(22) Filed: Feb. 20, 2001

(30) Foreign Application Priority Data
Feb. 22, 2000 (DE) 10007947.4-41

(51) Int. Cl. A61K 7/11

(52) U.S. Cl. 424/70.15

(57) ABSTRACT

The gel and the thickened hair treatment composition contain a combination of (A) at least one terpolymer of vinyl pyrrolidone, vinyl caprolactam and a basic acrylamide monomer, and (B) a gel-forming ingredient selected from the group consisting of gel formers which increase, or at most decrease by about 40%, a viscosity of an aqueous composition containing them when the at least one terpolymer is added to the aqueous composition. The preferred gel formers are acrylic acid polymers, xanthan gum and hydroxyalkylcellulose compounds.

### BACKGROUND OF THE INVENTION

1. Field of the Invention

The subject matter of the present invention is a gel containing a combination of a certain terpolymer and selected gel formers and a thickened hair treatment composition containing the gel of the described combination.

2. Prior Art

Hair treatment compositions, among others, in the form of thickened preparations, e.g. thickened fixing lotions, gels, liquid gels, spray gels, etc are used in order to fix and hold human hair or to stabilize a hair-do or hairstyle. This type of product usually contains a combination of a gel former and hair-fixing polymer. The cosmetic, hair-fixing polymers normally used for this purpose have good fixing properties in aqueous, alcoholic or aqueous-alcoholic solutions, which shape and fix the hair more or less well after application and stabilize the prepared hairstyle. However this is frequently associated with an unnatural stiff feel of the hair and the elasticity of the polymer film or the polymeric cross-linked hair-do is insufficient.

Terpolymers of vinyl pyrrolidone, vinyl caprolactam and 3-(N-dimethylaminopropyl)methacrylamide are known from WO 96/19971. The use of these polymers in fixing compositions, especially in aerosol and pump sprays, is also known. These polymers are especially suitable for use in aqueous spray formulations with a reduced content of readily volatile organic ingredients (low VOC sprays). These polymers have good fixing properties, but also the disadvantage that they definitely reduce the viscosity of the composition when used in gel form compositions in combination with a series of conventional thickeners. In this case either the concentration of the thickeners used must be definitely increased, which is undesirable because of cost and application engineering reasons, or the composition must be left with a less suitable, i.e. thinner, consistency.

### SUMMARY OF THE INVENTION

It is an object of the present invention to provide a thickened hair treatment composition that does not have the above-described disadvantages.

It is also an object of the present invention to provide a gel for preparing the thickened hair treatment composition.

It has now been found that these objects are attained by a gel containing a combination of

(A) at least one terpolymer of vinyl pyrrolidone, vinyl caprolactam and a basic acrylamide monomer; and

(B) a gel-forming ingredient selected from the gel formers which increase, or at most decrease by about 40%, preferably only 30%, the viscosity of a composition containing them when the ingredient (A) is added to the composition.

It is also attained by a thickened hair treatment composition containing the above-described combination of ingredients (A) and (B).

The terpolymer (A) is contained in the composition or gel according to the invention in an amount of from 0.01 to 20, preferably from 0.05 to 10, and especially preferably from 0.1 to 5, percent by weight, and the gel former is present in an amount of from 0.01 to 20, preferably from 0.05 to 10 and especially preferably from 0.1 to 5, percent by weight.

### DETAILED DESCRIPTION OF THE INVENTION

Suitable terpolymers (A) include those in which the acrylamide monomer is selected from the group consisting of dialkylaminoalkylmethacrylamides having alkyl groups, each containing one to four carbon atoms, and dialkylaminoalkylacrylamides having alkyl groups, each containing one to four carbon atoms. Dimethylaminopropylmethacrylamide is especially preferred. A method of making a polymer of this type is described in WO 96/19971. This polymer can be obtained commercially under the trademark AQUAFLEX® SF 40 (ISP) (INCI-name: PVP/vinylcaprolactam/DMAPA acrylates copolymer).

Suitable gel-formers or gel-forming ingredients (B) are, for example, Carbomers (acrylic acid polymers), especially those of the type Acrisint® 400, Xanthan gum or hydroxyalkylcellulose compounds, such as hydroxyethyl or hydroxypropylcellulose.

In a preferred embodiment the hair treatment composition according to the invention also contains from 0.01 to 15, preferably from 0.5 to 10,% by weight of at least one film-forming hair-fixing or hair-care polymer. This hair-fixing or hair-care polymer can be a nonionic, anionic, cationic, zwitterionic or amphoteric polymer. It can be a synthetic or natural polymer. The term “natural polymer” also includes chemically modified polymers of natural origin. Those polymers are especially preferred, which have sufficient solubility in alcohol or water/alcohol mixtures, in order to be present in the composition according to the invention in completely soluble form. The term “film-forming” polymer means those polymers which when used in a 0.01 to 5% aqueous, alcoholic or aqueous-alcoholic solution are in a position, to be deposited on the hair in a polymer film.

Suitable synthetic, nonionic film-forming, hair-fixing polymers are homo-polymers and copolymers, which are built up from at least one of the following monomers: vinyl pyrrolidone, vinyl caprolcactam, vinyl esters, such as vinyl acetate, vinyl alcohol, acrylamide, methacrylamide, alkyl- and dialkylacrylamides, alkyl- and dialkylmetharcylamides, alkylacrylates, alkylmethacrylates, propylene glycol or ethylene glycol. The alkyl groups in these monomers preferably have from one to seven carbon atoms, especially preferably from one to three carbon atoms. Suitable nonionic film-forming hair-fixing polymers are, for example, homopolymers of vinylcaprolactam, of vinyl pyrrolidone or of N-vinylformamide. Additional suitable synthetic film-forming, nonionic, hair-fixing polymers are, for example, copolymerizates of vinyl pyrrolidone and vinyl acetate, terpolymers of vinyl pyrrolidone, vinyl acetate and vinyl propionate, polyacrylamides, which are sold, for example, under the trademark AKYPOMINE®P 191 of CHEM-Y, Emmerich, or SEPIGEL®305 of Seppic; polyvinyl alcohols, which are sold for example under the trademark ELVANOL® of Dupont or VINOL® 523/540 of Air Products and polyethylene glycol/polypropylene glycol copolymers, which are sold, for example, under the trademark UCON® of Union Carbide. Polyvinyl pyrrolidone and polyvinyl pyrrolidone/vinyl acetate copolymers are especially preferred.

Suitable anionic film-forming polymers include synthetic homo- or copolymers with neutralizable monomer units containing acid groups, which are co-polymerizable with co-monomers, if necessary, which contain no acid groups. The acid groups are preferably selected from the group consisting of —COOH, —SO₃H, —OSO₃H, —OPO₂H and —OPO₃H₂ groups, of which the carboxylic acid group is particularly preferred. The acid groups can be not-neutralized, partially neutralized or completely neutralized. They are preferably present in an anionic form, neutralized from 50 to 100%, or in a completely neutralized form. Organic or inorganic bases suitable for cosmetic applications, for example, amino-alcohols, such as aminomethylpropanol (AMP), triethanolamine, monoethanol-amine or tetrahydroxypropylethylene diamine and ammonia, NaOH and others, can be used as neutralizing agent. Suitable monomers are unsaturated, radically polymerizable compounds, which have at least one acid group, especially carboxyvinyl monomers. Monomers containing suitable acid groups include, for example, acrylic acid, methacrylic acid, crotonic acid, maleic acid or maleic acid anhydride, aldehydocarboxylic acids or ketocarboxylic acids.

The comonomers not substituted with acid groups include, e.g., acryl amide, methacrylamides, alkyl and dialkylacrylamides, alkyl and dialkylmethacrylamides, alkylacrylates, alkylmethacrylates, vinylcaprolactone, vinylpyrrolidone, vinyl esters, vinyl alcohols, propylene glycol or ethylene glycol, amine-substituted vinyl monomers, such as dialkylaminoalkylacrylates, dialkylaminoalkylmethacrylates, monoalkyl-aminoalkylacrylates and monoalkylaminoalkylmethacrylates, in which the alkyl groups of these monomers preferably contain one to seven carbon atoms, especially preferably from one to three carbon atoms.

Suitable anionic polymers with acid groups are particularly copolymers of acrylic acid or methacrylic acid with monomers selected from the group consisting of acrylic acid or methacrylic acid esters, acryl amides, methacrylamides and vinylpyrrolidones, homopolymers of crotonic acid and copolymers of crotonic acid with monomers selected from the group consisting of vinyl esters, acrylic acid or methacrylic acid esters, acrylamides and methacrylamides. A suitable natural polymer is, for example, shellac.

Preferred polymers with acid groups include cross-linked or uncross-linked vinyl acetate/crotonic acid copolymers. Similarly the copolymers of methyl vinyl ether and maleic acid anhydride are especially preferred. Additional suitable anionic polymers include for example terpolymers of acrylic acid, alkyl acrylate and N-alkylacrylamide, especially acrylic acid/ethyl acrylate/N-t-butylacrylamide terpolymer or terpolymers of vinyl acetate, crotonate and vinyl alkanoate, especially vinyl acetate/crotonatelvinyl neodecanoate copolymers.

Suitable film-forming amphoteric polymers are those polymers, which contain both basic or cationic groups, such as primary, secondary, tertiary or quaternary amines, as additional functional groups besides acidic or anionic groups. Suitable copolymers are formed, for example, from alkylacrylamides (especially octylacrylamide), alkylaminoalkylmethacrylates (especially t-butylaminoethylmethacrylate) and two or more monomers, comprising acrylic acid, methacrylic acid or their esters, such as the copolymer made from acrylic acid, methacrylic acid or its ester, e.g. sold under the trademark AMPHOMER® or AMPHOMER® LV-71 of National Starch, USA. Additional examples of these comonomers suitable as ingredient (B) include copolymers of acrylic acid, methacrylate and methacrylamidopropyltrimethylammonium chloride (INCI: polyquaternium-47), such as that sold by Calgon under the trademark MERQUAT® 2001; copolymers made from acrylamidopropyltrimethylammonium chloride and acrylates marketed by Stockhausen under the tradename W 37194 or copolymers made from acrylamide, acrylamidopropyltrimonium chloride, 2-amidopropylacrylamide sulfonate and dimethylaminopropylamine (INCI: Polyquaternium-43), as sold for example by Societe Francaise Hoechst under the trademark BOZEQUAT® 4000. Polymers made with monomers containing betaine groups, such as copolymers from methacryloylethylbetaine and two or more monomers of acrylic acid or their simple esters, having the INCI name methacryloyl ethyl betaine/acrylates copolymers, are also suitable. The zwitterionic amphoteric polymers, which are described in JP 10-29919 or JP 10-25344, are also suitable.

Suitable cationic hair-fixing polymers are polymers that are cationic or contain basic, i.e. cationizable groups. These polymers contain nitrogen-containing groups, such as primary, secondary or tertiary amines. The basic groups are contained in polymer chains or preferably are substituents on one or more monomers. The polymers with the basic groups can be natural or synthetic homopolymers or copolymers with amine-substituted monomer units and, if necessary, with non-basic comonomers. Suitable polymers with basic groups are, for example, copolymers of amine-substituted vinyl monomers and non-amine substituted monomers. Amine-substituted vinyl monomers are, for example, dialkylaminoalkylacrylates, dialkylaminoalkylmethacrylates, monoalkylaminoalkylacrylates and monoalkylaminoalkylmethacrylates. The alkyl groups of these monomers are preferably lower alkyl groups, such as alkyl groups having one to seven carbon atoms, especially preferably from one to three carbon atoms.

Suitable non-amine substituted co-monomers include, for example, acrylamides, methacrylamides, alkyl- and dialkylacrylamides, alkyl- and dialkylmethacryl-amides, alkylacrylates, alkylmethacrylates, vinylcaprolactones, vinylpyrrolidones, vinyl esters, vinyl alcohols, maleic acid anhydrides, propylene glycol or ethylene glycol, in which the alkyl groups preferably have one to seven carbon atoms, especially one to three carbon atoms.

Suitable polymers with cationic groups preferably contain quaternary amine groups. The cationic polymers can be homo- or copolymer compounds, in which the quaternary nitrogen groups either are in polymer chains or preferably contained as substituents on one or more monomers. The monomers containing the ammonium groups can be copolymerized with the above-described non-amine substituted monomers. Suitable ammonium-substituted vinyl monomers are, for example, trialkylmethacryloxyalkyl ammonium groups, trialkylacryl-oxyalkyl ammonium groups, dialkyldiallyammonium groups and quaternary vinyl ammonium monomer groups with cyclic, cationic nitrogen-containing groups, such as pyridinium imidazolium or quaternary pyrrolidones, e.g. alkylvinylimidizolium, alkylvinylpyridinium or alkylvinylpyrrolidone salts. The alkyl groups of these monomers are preferably lower alkyl groups, such as alkyl groups having one to seven carbon atoms, especially preferably from one to three carbon atoms.

Suitable polymers with quaternary amine groups are, for example, the polymers described with the tradename, polyquaternium, in the CTFA Cosmetic Ingredient Dictionary, such as methylvinylimidazolium chloride/vinylpyrrolidone copolymer (polyquaternium-16) or quaternized vinyl pyrrolidone/dimethyl-aminoethyl-methacrylate copolymer (polyquaternium-11), homopolymers and copolymers of dimethyidiallylammonium chloride (polyquaternium-6 and -7), quaternarized hydroxyethylcellulose (Polyquaternium-10) or quaternized guar derivative compounds.

Polyvinylpyrrolidone/dimethylaminoethylmethacrylate copolymer is an especially suitable cationic film-forming polymer, which can be included in the composition according to the invention. Additional cationic polymers include, for example, the copolymer of polyvinylpyrrolidone and imidazolimin-methochloride, the terpolymer of dimethyldiallylammonium chloride, sodium acrylate and acrylamide, the terpolymer of vinyl pyrrolidone, dimethylamino-ethylmethacrylate and vinylcaprolactam, quaternarized ammonium salt of hydroxyethyl cellulose and a trimethylammonium-substituted epoxide compound and vinylpyrrolidone/methacrylamidopropyltrimethylammonium chloride copolymer.

The composition according to the invention is provided preferably in an aqueous, an alcoholic or in an aqueous-alcoholic medium with preferably at least ten percent by weight water and a maximum of 40% by weight alcohol. The lower alcohols, which are normally used for cosmetic purposes, with 1 to 4 carbon atoms, especially ethanol and isopropanol, are especially suitable as the alcohols. The composition according to the invention can have a pH of from 2.0 to 9.5. A pH in a range of from 5 to 8 is especially preferred. Organic solvents with a boiling point under 400° C., or mixtures of these solvents, can be present in the composition according to the invention in an amount of from 0.1 to 15%, preferably 1 to 10, percent by weight as additional co-solvents. Glycerol, ethylene glycol and propylene glycol in amounts up to 20 percent by weight are especially preferred as the additional co-solvents.

Furthermore the composition according to the invention can contain conventional additive ingredients usually used for hair treatment composition, for example wetting agents and emulsifiers from the classes of anionic, cationic, nonionic or amphoteric surface active substances, such as fatty alcohol sulfates, alkyl-benzene sulfonates, alkyltrimethyl ammonium salts, alkyl betaines, in an amount of from 0.1 to 15 percent by weight; moisturizing agents; perfume oils, in an amount of 0.1 to 1 percent by weight; turbidity-inducing agents, for example ethylene glycol distearates, in an amount of about 0.2 to 5.0 percent by weight; pearlescence-imparting agents, such as a mixture of fatty acid monoalkylol-amides and ethylene glycol distearate, in an amount of about 1.0 to 10 percent by weight; bactericides and fungicides, for example 2,4,4-trichloro-2-hydroxy-diphenyl ether or methyl chlorisothiazolione, in an amount of from 0.01 to 1.0 percent by weight; thickeners, such as coconut oil fatty acid diethanol amides, in an amount of from about 0.2 to 3.0 percent by weight; buffer substances, such as sodium citrate or sodium phosphate, in an amount of from 0.1 to 1.0 percent by weight; dye substances, such as fluorescein sodium salt, in an amount of from about 0.1 to 1.0 percent by weight; care substances, such as plant and vegetable extracts, protein and silk hydrolyzates, lanolin derivative compounds, in an amount of from 0.1 to 5 percent by weight; physiologically compatible silicone derivative compounds, such as volatile or non-volatile silicones or high molecular weight siloxane polymers, in an amount of from 0.05 to 20 percent by weight; light-protective ingredients, antioxidants, radical-trapping agents, anti-flaking agents, in an amount of from about 0.01 to 4 percent by weight; fatty alcohols, luster-imparting substances, vitamins, softeners, combability improving substances, defatting agents and antifoaming agents.

The composition according to the invention can be provided in different application forms, for example as a gel, liquid-gel, spray-gel, as a thickened lotion or as a gel cream and/or as a turbid or cloudy gel. However a clear or transparent gel is the preferred form. If the hair treatment composition is a hair gel, the viscosity of the gel is preferably from 500 to 50,000 cSt, especially preferably from 1,000 to 15,000 cSt as measured in a dynamic viscosity measurement with a HAAKE rotary viscometer at a temperature of 25° C., measuring body, Haake SV, DIN, setting 8, speed 5.

The following examples should illustrated the subject matter of the invention without limiting the scope of the appended claims. The polymer content described in the examples relates to the solids content.

### EXAMPLES

### Example 1

### Comparative Example

Viscosities were measured for purely aqueous composition with a content of different gel formers with and without addition of 5 percent by weight AQUAFLEX®SF 40 (40% solution of PVP/vinyl caprolactam/DMAPA acrylates copolymer in ethanol). The conditions for the viscosity measurement were: temperature 25° C.

Measurement Unit: HAAKE rotary viscometer, measurement body, Haake SV DIN, setting 8, speed 5. The results of the measurements are summarized in Table I. The % in the table means the percentage by weight of the ingredient in the composition.

The results in Table I show that the viscosity of the compositions is increased less than 40% when the 5% AQUAFLEX®SF is added.

TABLE I
VISCOSITIES OF AQUEOUS GEL FORMER COMPOSITIONS
WITH AND WITHOUT ADDITION OF 5% by weight
Aquaflex ® SF 40

| | Viscosity (Pa s) | Viscosity (Pa s) | |
|---|---|---|---|
| | without | with | Δ |
| Composition | Aquaflex ® SF 40 | Aquaflex ® SF 40 | [%] |
| Carbopol ® 980, | 13.5 | 3.5 | −74 |
| 0.35%; AMP 0.26% | | | |
| Acrisint ® 400, | 22.5 | 17 | −24 |
| 0.6%; AMP, 0.5% | | | |
| Pemulen ® TR1¹⁾, | 2.9 | 0.744 | −74 |
| 0.13%; AMP, 0.16% | | | |
| Xanthan Gum, 2% | 1.2 | 2.3 | +92 |
| Karaya Gum, 2% | 2.08 | 0.028 | −99 |
| Lambda Carrageenan | 2.3 | 1.3 | −43 |
| 2% | | | |
| Structure ® 3001²⁾, | 10.1 | 5.3 | −48 |
| 10% | | | |
| Hydroxyethylcellulose | 6.15 | 6.68 | +9 |
| 2% | | | |
| Structure ® Plus³⁾, | >100 | 13.7 | <−87 |
| 30%; formic acid, | | | |
| 0.25% | | | |
| Synthalen ® W 2000⁴⁾ | 1.8 | 1.1 | −39 |
| 6.5%; AMP, 0.645% | | | |

¹⁾acrylates/C₁₀-C₃₀ alkyl acrylates cross-polymer
²⁾acrylates/Ceteth-20 Itaconate copolymer
³⁾acrylates/aminoacrylates copolymer
⁴⁾acrylates/C₁₂-C₁₄ Pareth-25 acrylate copolymer

In the following exemplary compositions the amount of the raw material Aquaflex® SF 40 relates to the solids content of the polymer.

### Example 2

### Liquid Hair Gel

| | |
|---|---|
| 1.0 g | Aquaflex ® SF 40 (PVP/Vinyl Caprolactam/ |
| | DMAPA Acrylates Copolymer) |
| 1.5 g | Dow Corning Surfactant 193 (Dimethicone copolyol) |
| 0.3 g | Acrisint ® 400 |
| 0.3 g | Aminomethylpropanol 95% |
| 0.15 g | Perfume |
| 0.2 g | PEG-40 hydrogenated castor oil |
| 0.2 g | PPG-1-PEG-9 Lauryl Glycol Ether |
| 0.4 g | Hydroxyethyl cellulose |
| 16.5 g | Ethanol |
| to 100 g | water |

### Example 3

### Quick-Drying Hair Gel

| | |
|---|---|
| 2.8 g | Aquaflex ® SF 40 (PVP/Vinyl Caprolactam/ |
| | DMAPA Acrylates Copolymer) |
| 1.5 g | Dow Corning Surfactant 193 (Dimethicone copolyol) |
| 0.8 g | Acrisint ® 400 |
| 0.62 g | Aminomethylpropanol 95% |
| 0.15 g | Perfume |
| 0.3 g | Panthenol |
| 34.2 g | Ethanol |
| to 100 g | water |

### Example 4

### Liquid Hair Gel

| | |
|---|---|
| 1.0 g | Aquaflex ®SF 40 (PVP/Vinyl Caprolactam/ |
| | DMAPA Acrylates Copolymer) |
| 1.0 g | Dow Corning Surfactant 193 (Dimethicone copolyol) |
| 2.5 g | Klucel ® HF (hydroxypropylcellulose) |
| 0.1 g | citric acid |
| 0.15 g | Perfume |
| 16.5 g | Ethanol |
| to 100 g | water |

### Example 5

### Hair Gel

| | |
|---|---|
| 1.05 g | Aquaflex ® SF 40 (PVP/Vinyl Caprolactam/ |
| | DMAPA Acrylates Copolymer) |
| 4.2 g | Sorbitol |
| 0.3 g | Acrisint ® 400 |
| 1.6 g | Aminomethylpropanol 95% |
| 0.2 g | Perfume |
| 1.5 g | PEG-25 PABA |
| 0.2 g | Phenoxyethanol |
| 0.05 g | Mica |
| 34.2 g | Ethanol |
| to 100 g | water |

### Example 6

### Hair Gel

| | |
|---|---|
| 3.0 g | Aquaflex ® SF 40 (PVP/Vinyl Caprolactam/ |
| | DMAPA Acrylates Copolymer) |
| 5.2 g | Glycerol |
| 4.0 g | Propylene glycol |
| 0.35 g | Carbomer |
| 0.3 g | Aminomethylpropanol 95% |
| 0.2 g | Perfume |
| 1.5 g | Polysorbate-40 |
| 0.2 g | Phenoxyethanol |
| 0.5 g | PEG-25 PABA |
| 4.5 g | Ethanol |
| to 100 g | water |

### Example 7

### Liquid Hair Gel

| | |
|---|---|
| 0.5 g | Aquaflex ® SF 40 (PVP/Vinyl Caprolactam/ |
| | DMAPA Acrylates Copolymer) |
| 7.0 g | Glucose |
| 3.8 g | Propylene glycol |
| 0.3 g | Carbomer |
| 0.3 g | Aminomethylpropanol 95% |
| 0.15 g | Perfume |
| 0.18 g | PEG-40 Hydrogenated Castor Oil |
| 0.18 g | PPG-1-PEG-9 Lauryl Glycol Ether |
| 0.2 g | Phenoxyethanol |
| 0.5 g | PEG-25 PABA |
| 16.5 g | Ethanol |
| to 100 g | water |

The disclosure in German Patent Application 10007947.4-41 of Feb. 22, 2001 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in a gel composition and a thickened hair treatment composition, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A gel containing a combination of
at least one terpolymer of vinyl pyrrolidone, vinyl caprolactam and a basic acrylamide monomer, and
a gel-forming ingredient selected from the group consisting of viscosity-determining gel formers;
wherein said viscosity-determining gel formers are only those compounds such that respective aqueous compositions containing said viscosity-determining gel formers have viscosities that increase, or at most decrease by about 40%, when said at least one terpolymer is added to the respective aqueous compositions.

2. The gel as defined in claim 1, wherein said basic acrylamide monomer is selected from the group consisting of dialkylaminoalkylmethacrylamides having alkyl groups each containing from one to four carbon atoms and dialkylamino-alkylacrylamides having alkyl groups each containing from one to four carbon atoms.

3. The gel as defined in claim 1, containing from 0.01 to 20 percent by weight of said at least one terpolymer and from 0.01 to 20 percent by weight of said gel-forming ingredient.

4. The gel as defined in claim 1, wherein said viscosity-determining gel formers are only those such that said respective aqueous compositions have viscosities that increase when said at least one terpolymer is added thereto.

5. A gel containing a combination of
at least one terpolymer of vinyl pyrrolidone, vinyl caprolactam and a basic acrylamide monomer, and
a gel-forming ingredient selected from the group consisting of acrylic acid polymers, xanthan gum and hydroxyalkylcellulose compounds.

6. The gel as defined in claim 5, wherein said hydroxyalkyl cellulose compounds include hydroxylethylcellulose and hydroxypropylcellulose.

7. The gel as defined in claim 5, wherein said basic acrylamide monomer is selected from the group consisting of dialkylaminoalkylmethacrylamides having alkyl groups each containing from one to four carbon atoms and dialkylaminoalkylacrylamides having alkyl groups each containing from one to four carbon atoms.

8. The gel as defined in claim 5, containing from 0.01 to 20 percent by weight of said at least one terpolymer and from 0.01 to 20 percent by weight of said gel-forming ingredient.

9. A hair treatment composition comprising a gel, said gel including a combination of at least one terpolymer of vinyl pyrrolidone, vinyl caprolactam and a basic acrylamide monomer, and a viscosity-determining gel-forming ingredient, wherein said viscosity-determining gel formers are only those compounds such that respective aqueous compositions containing said viscosity-determining gel formers have viscosities that increase, or at most decrease by about 40%, when said at least one terpolymer is added to the respective aqueous compositions.

10. The hair treatment composition as defined in claim 9, containing from 0.01 to 20 percent by weight of said at least one terpolymer and from 0.01 to 20 percent by weight of said gel-forming ingredient.

11. The hair treatment composition as defined in claim 9, further comprising from 0.01 to 15% by weight of at least one film-forming, hair-fixing polymer ingredient selected from the group consisting of non-ionic film-forming hair-fixing polymers, amphoteric film-forming hair-fixing polymers, cationic film-forming hair-fixing polymers and anionic film-forming hair fixing polymers.

12. A hair treatment composition comprising a gel, said gel including a combination of at least one terpolymer of vinyl pyrrolidone, vinyl caprolactam and a basic acrylamide monomer, and a viscosity-determining gel-forming ingredient, said viscosity-determining gel-forming ingredient being selected from the group consisting of acrylic acid polymers, xanthan gum and hydroxyalkylcellulose compounds.

13. The hair treatment composition as defined in claim 12, containing from 0.01 to 20 percent by weight of said at least one terpolymer and from 0.01 to 20 percent by weight of said gel-forming ingredient.

14. The hair treatment composition as defined in claim 12, further comprising from 0.01 to 15% by weight of at least one film-forming, hair-fixing polymer ingredient selected from the group consisting of non-ionic film-forming hair-fixing polymers, amphoteric film-forming hair-fixing polymers, cationic film-forming hair-fixing polymers and anionic film-forming hair fixing polymers.

15. The hair treatment composition as defined in claim 12, wherein said hydroxyalkylcellulose compounds include hydroxylethylcellulose and hydroxypropylcellulose.

16. The hair treatment composition as defined in claim 9 or 12, further comprising water and at least one cosmetic additive ingredient selected from the group consisting of alcohols, nonionic surfactants, cationic surfactants, anionic surfactants, amphoteric surfactants, moisturizing agents, perfume oils, turbidity-inducing agents, pearlescence-imparting agents, bactericides, fungicides, thickeners, buffer substances, dye substances, care substances, silicone derivative compounds, light-protective agents, fatty alcohols, luster-imparting substances, vitamins, softeners, combability-improving substances, defatting agents and antifoaming agents.

17. The hair treatment composition as defined in claim 9 or 12, having a pH of from 2.0 to 9.5 and further comprising at least one member selected from the group consisting of water and alcohols.
